(19) United States
(12) Patent Application Publication
(10) Pub. No.: US 2004/0152954 A1
(43) Pub. Date: Aug. 5, 2004

(54) Menu-driven medical device configuration

(76) Inventors: Christopher Pearce, Monroe, WA (US); Michael D. McMahon, Lake Forest Park, WA (US); Maria W.T. de Louw, Schayk (NL); Annemieke A.J. Heutinck, s'Hertogenbosch (NL)

Correspondence Address: SHUMAKER & SIEFFERT, P. A., 8425 SEASONS PARKWAY, SUITE 105, ST. PAUL, MN, 55125, US

(21) Appl. No.: 10/357,319

(22) Filed: Jan. 31, 2003

(51) Int. Cl. G09B 23/28; A61B 5/00

(52) U.S. Cl. 600/300; 434/262

(57) ABSTRACT

The invention is directed to techniques for configuration of a medical device, such as a defibrillator or patient monitor. The configuration is menu-driven. The medical device may present a menu of patient conditions to an operator. When the operator selects a patient condition from the menu, the device selects a configuration parameter and self-configures with the configuration parameter. The menu-driven techniques may further allow an operator to change the configuration of the device to configure the device more specifically to the condition of the patient. The menu-driven techniques may also allow the device to self-configure with a default configuration parameter in the event the operator fails to make a menu selection.

### FIELD

The invention relates to medical devices, and more particularly, to medical devices that monitor or treat medical conditions.

### BACKGROUND

Emergency medical technicians (EMTs) save lives every day by responding to emergencies. EMTs provide immediate medical attention to a patient. Medical attention may include, for example, determining the nature and extent of the condition of the patient and administering therapy.

In some situations, an EMT may arrive on the scene of an emergency without knowing all of the details of the emergency. The EMT may be unaware, for example, of whether the patient is an adult or a child. Adult patients and pediatric patients may often be monitored and treated in different ways.

The EMT may also be unaware of the nature of the medical emergency. The patient may have suffered trauma, or a stroke, or ventricular fibrillation, or some other medical problem, but the EMT may be unable to determine the nature of the problem until the EMT arrives on the scene. Appropriate emergency care depends upon the nature of the emergency.

### SUMMARY

A medical device, such as a defibrillator or patient monitor, may be used to diagnose, monitor or treat a variety of medical conditions at the scene of an emergency. In general, the invention is directed to techniques for configuring the medical device to apply to the specific medical condition of a patient. The configuration is menu-driven. The configuration may be initiated by an operator at the scene of an emergency, and may be changes as circumstances warrant.

In one embodiment, the invention is directed to a method comprising presenting a menu of patient conditions to an operator. The operator may or may not make a selection of a patient condition from the menu. When the operator makes such a selection, the method includes receiving the selection, selecting a configuration parameter, and configuring a medical device with the configuration parameter. When the operator fails to make a selection within a particular time period, the method may comprise selecting a generic configuration parameter, and configuring a medical device with the generic configuration parameter.

In another embodiment, the invention is directed to a method comprising presenting a menu of patient conditions, receiving from an operator a selection of a patient condition from the menu, selecting at least one configuration parameter as a function of the selection of the patient condition from the menu and configuring a medical device with the configuration parameter. This embodiment may include the presentation of more than one menu. A second menu may be presented, for example, to allow for a more detailed description of the patient conditions, or to allow the medical device to be reconfigured for another patient condition.

The invention further includes computer-readable media comprising instructions for causing a programmable processor to carry out the methods described above.

In a further embodiment of the invention, the invention is directed to a device that includes an output device to present a menu of patient conditions to an operator, an input device to receive a selection of a patient condition from the menu, and a processor to select at least one configuration parameter and to configure the medical device with the configuration parameter. The device may be a defibrillator or a patient monitor, but the invention is not limited to those kinds of devices.

The invention may offer one or more advantages. Menu-driven configuration of a medical device may provide quick and easy configuration of a medical device to the particular patient conditions at hand. The configuration may be as general or as specific as desired. An operator may configure the device while responding to the emergency, and may reconfigure the device in response to developments in the emergency. In addition, the device may include a default self-configuration with one or more generic configuration parameters, making the device ready to handle typical emergencies.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a schematic view of a defibrillator that may be used to practice the techniques of the invention.

FIG. 2 is a schematic view of a patient monitor that may be used to practice the techniques of the invention.

FIG. 3 is an example of a screen display that presents a menu of patient conditions.

FIG. 4 is an example of a screen display that has been configured according to configuration parameters, and that also presents a menu of patient conditions in accordance with the invention.

FIG. 5 is a flow diagram illustrating menu-driven medical device configuration techniques in accordance with the invention.

### DETAILED DESCRIPTION

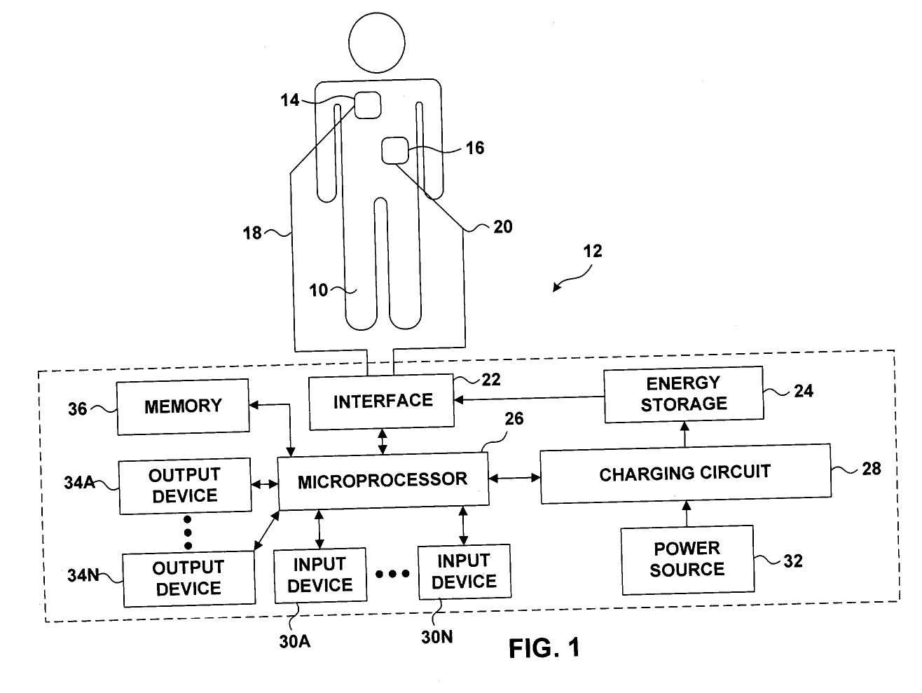

FIG. 1 is a block diagram showing a patient 10 coupled to an external defibrillator 12. External defibrillator 12 is one example of a device that may be used to practice the invention. Defibrillator 12 administers defibrillation therapy to patient 10 via electrodes 14 and 16, which may be hand-held electrode paddles or adhesive electrode pads placed on the skin of patient 10. The body of patient 10 provides an electrical path between electrodes 14 and 16.

Electrodes 14 and 16 are coupled to defibrillator 12 via conductors 18 and 20 and interface 22. In a typical application, interface 22 includes a receptacle, and connectors 18, 20 plug into the receptacle. Electrical impulses or signals may be sensed by defibrillator 12 via electrodes 14 and 16 and interface 22. Electrical impulses or signals may also be delivered from defibrillator 12 to patient 10 via electrodes 14 and 16 and interface 22.

Interface 22 includes a switch (not shown in FIG. 1) that, when activated, couples an energy storage device 24 to electrodes 14 and 16. Energy storage device 24 stores the energy for a dosage of energy or current to be delivered to patient 10. The switch may be of conventional design and may be formed, for example, of electrically operated relays. Alternatively, the switch may comprise an arrangement of solid-state devices such as silicon-controlled rectifiers or insulated gate bipolar transistors.

Energy storage device 24 includes components, such one or more capacitors, that store the energy to be delivered to patient 10 via electrodes 14 and 16. Before a defibrillation pulse may be delivered to patient 10, energy storage device 24 must be charged. A microprocessor 26 directs a charging circuit 28 to charge energy storage device 24 to a high voltage level. Microprocessor 26 may automatically direct charging circuit 28 to begin charging, or microprocessor 26 may direct charging circuit 28 to begin charging upon the instruction of an operator such as an EMT. An operator may instruct microprocessor with one or more input devices 30A-30N (hereinafter 30), such as one or more buttons, a keyboard, a touch screen, a voice recognition module or a pointing tool.

Charging circuit 28 comprises, for example, a flyback charger that transfers energy from a power source 32 to energy storage device 24. Because the life of patient 10 may depend upon receiving defibrillation, charging should take place rapidly so that the defibrillation shock may be delivered with little delay.

When the energy stored in energy storage device 24 reaches the desired level, defibrillator 12 is ready to deliver the defibrillation shock. The shock may be delivered automatically or manually. Defibrillator 12 may notify the operator that charging is completed using one or more output devices 34A-34N (hereinafter 34), such as a display screen, an audible sound generator, a voice synthesizer, a printer or an indicator light. In the case of a manual delivery, microprocessor 26 may activate an output device 34 that informs the operator that defibrillator 12 is ready to deliver a defibrillation shock to patient 10. The operator may activate the switch by manually operating an input device 30, such as by pressing a button. Defibrillator 12 delivers a defibrillation shock to patient 10.

The goal of defibrillation is to depolarize the heart with electrical current and cause the heart to reestablish a normal sinus rhythm. In some patients, one shock is insufficient to reestablish normal rhythm, and one or more additional defibrillation shocks may be required. Before another shock may be administered, however, charging circuit 28 ordinarily must transfer energy from power source 30 to energy storage device 24, thereby recharging energy storage device 24. In recharging energy storage device 24, as in the initial charging, time is of the essence, and charging circuit 28 therefore charges energy storage device 24 quickly.

Microprocessor 26 may regulate charging circuit 28 to store a particular level of energy in energy storage device 24. The level of energy stored may be important because the energy or current dosage delivered to patient 10 need not be the same in each shock. In addition, the energy or current dosage delivered to patient 10 when patient 10 is an adult may be different than when patient 10 is a pediatric patient. The energy delivered to a pediatric patient in defibrillation therapy is considerably less than that delivered to an adult patient.

Power source 30 may comprise, for example, batteries and/or an adapter to an exterior power source such as an electrical outlet. In addition to supplying energy to charging circuit 28 and energy storage device 24, power source 30 also supplies power to components such as microprocessor 26, input devices 30 and output devices 34, e.g., via a power supply circuit (not shown in FIG. 1).

In addition to controlling the level of energy of a defibrillation pulse and the delivery of the defibrillation pulse, microprocessor 26 may also modulate the electrical pulse delivered to patient 10. Microprocessor 26 may, for example, regulate the shape of the waveform of the electrical pulse and the duration of the pulse. Microprocessor 26 may also monitor electrocardiogram (ECG) signals sensed via electrodes 14 and 16 and received via interface 22. Microprocessor 26 may display these signals via an output device 34 such as a display screen or printer. In some embodiments, microprocessor 26 may also analyze the ECG signals and determine whether patient 10 suffers from a condition that requires a defibrillation shock. In addition, microprocessor 26 may evaluate the efficacy of an administered defibrillation shock.

Furthermore, microprocessor 26 may store and retrieve data from memory 36. Memory 36 may include volatile storage, such as random access memory, and/or non-volatile storage, such as Flash memory or a hard disk. Memory 36 stores instructions that direct the operation of microprocessor 26. In addition, memory 36 stores information about patient 10 and defibrillator 12. For example, memory 36 may store the ECG of patient 10, information about the number of shocks delivered to patient 10, the energy delivered per shock, the timing of shocks and the patient response to shocks.

As will be described below, defibrillator 12 may be configured to one or more patient conditions. Output device 34 may present a menu of patient conditions to the operator, and input device 30 may receive a selection of a patient condition from the menu. Microprocessor 26 may select a configuration parameter as a function of the selection by the operator, if any. When the operator selects a patient condition from the menu, microprocessor 26 may select one or more configuration parameters as a function of the selection by the operator. When the operator fails to make a selection, microprocessor 26 may select a generic configuration parameter. Microprocessor 26 configures defibrillator 12 with the configuration parameter.

The configuration may be illustrated by an example. Defibrillator 12 may present the operator with a menu of patient conditions, such as “cardiac,” “trauma” and “pediatric.” Defibrillator 12 may display the menu on an output device such as a display screen. The operator, finding that patient 10 is a child, selects “pediatric” from the menu. The operator may perform the selection by interacting with an input device such as a button. Upon receiving the selection from the menu, defibrillator 12 self-configures as a function of the patient condition. Microprocessor 26 may, for example, regulate charging circuit 28 to store a particular level of energy in energy storage device 24 suitable for a pediatric patient, because the energy needed to defibrillate a pediatric patient is considerably less than the energy needed to defibrillate an adult patient. Microprocessor 26 may also retrieve from memory 36 other information that pertains to treatment of a pediatric patient, such as a pediatric care protocol.

In some circumstances, the operator may interact with the menu by making no selection at all. In other words, defibrillator 12 may display the menu on an output device for a time interval, without any selection from the menu during that interval. In such a case, defibrillator may select one or more configuration parameters without receiving a selection from an operator. Such “generic configuration parameters” may be used by microprocessor 26 to configure defibrillator 12. Generic configuration parameters may be default parameters suited to general, non-specific emergencies. By selecting a generic configuration parameter, microprocessor 26 does not necessarily prevent the operator from selecting a patient condition at a later time, but microprocessor 26 does configure itself to provide some default monitoring or therapy functions when the operator makes no selection.

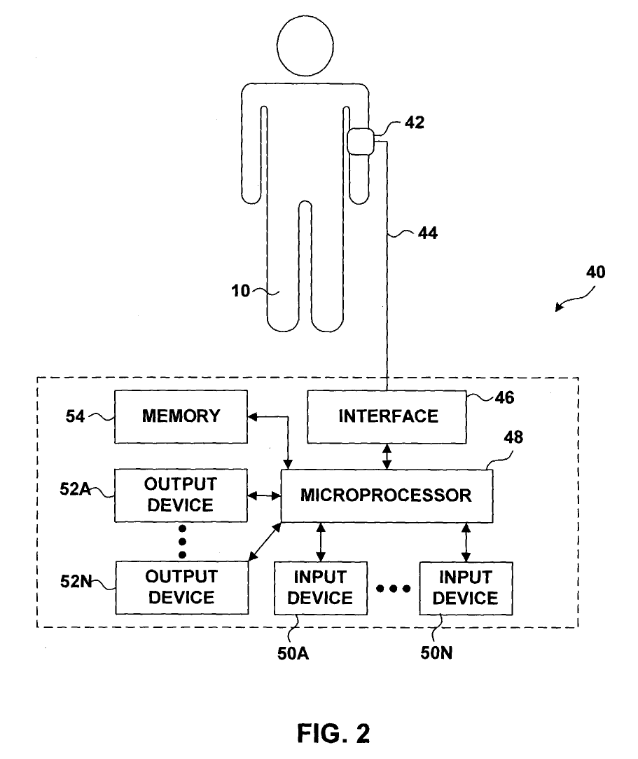

FIG. 2 is a block diagram showing patient 10 coupled to a patient monitor 40. Monitor 40 is another example of a device that may be used to practice the invention. Monitor 40 senses a patient condition with a sensor 42 coupled to monitor 40 via link 44. Sensed patient conditions may include, for example, body temperature, blood pressure, blood oxygen levels, respiration, patient impedance, heart rate, heart rhythm, expired carbon dioxide concentration and the like. Monitor 40 does not, however, administer therapy to patient 10.

Monitor 40 is similar to defibrillator 12 in several respects. Monitor 40 may include an interface 46 that receives link 44. Electrical impulses or other signals sensed by sensor 42 may be received by monitor 40 through interface 46. Monitor 40 also includes a microprocessor 48, which processes the received signals.

An operator may interact with monitor 40 with one or more input devices 50A-50N (hereinafter 50), such as one or more buttons, a keyboard, a touch screen, a voice recognition module or a pointing tool, and one or more output devices 52A-52N (hereinafter 52), such as a display screen, an audible sound generator, a voice synthesizer, a printer or an indicator light. Monitor 40 may also include memory 54, which may store data and instructions for microprocessor 48. Like memory 36 in defibrillator 12, memory 54 may store information about patient 10 and therapy provided to patient 10.

Like defibrillator 12, monitor 40 may be configured to one or more patient conditions. Output device 52 may present a menu of patient conditions to the operator, and input device 50 may receive a selection of a patient condition from the menu. Microprocessor 48 may select a configuration parameter as a function of the selection by the operator, if any. When the operator selects a patient condition from the menu, microprocessor 48 may select one or more configuration parameters as a function of the selection by the operator. When the operator fails to make a selection, microprocessor 48 may select a generic configuration parameter. Microprocessor 48 configures monitor 40 with the configuration parameter.

In the exemplary case when monitor 40 presents the operator with a menu of patient conditions and the operator selects “pediatric,” monitor 40 may self-configure to monitor a pediatric patient. Microprocessor 48 may, for example, set alarm parameters suitable for a pediatric patient. The heart rate, heart rhythm and respiration for a pediatric patient may be substantially different than for an adult patient. Patient data that would be abnormal and cause for alarm with an adult patient may be normal for a pediatric patient, and vice versa.

Configuration of defibrillator 12 or monitor 40 may further include selection and implementation of a medical protocol. As used herein, “protocol” is defined broadly. “Protocol” encompasses procedures for treating patients, including plans, guidelines and rules for treating patients. The term encompasses general procedures, as well as procedures applicable to a specific patient complaint, condition or presentation. “Protocol” further includes rules and guidelines applicable to a jurisdiction, such as treatment procedures adopted by a regulating authority responsible for overseeing EMTs. A regulating authority may be, for example, a regional health care system administrator or a government administrative agency that licenses and regulates EMTs.

The procedures of a protocol may be embodied as a checklist, a questionnaire, a flow diagram, a series of notices or the like. As used herein, “protocol” includes the embodiment of the procedures, as well as what an operator should do in carrying out the procedures.

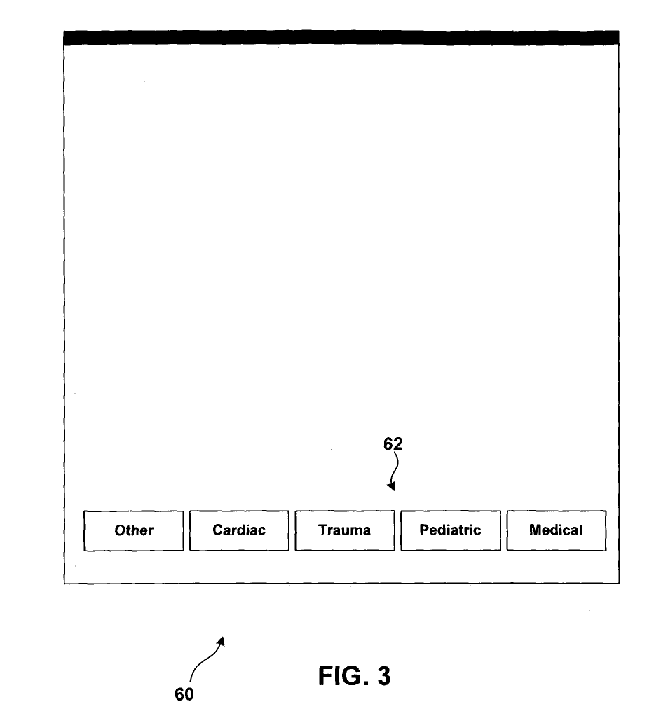

FIG. 3 is an example of a screen display 60 that presents a menu 62 of patient conditions to an operator by an output device such as a display screen or touchscreen. A medical device such as defibrillator 12 or monitor 40 may present menu 62 upon activation of the device. An operator such as an EMT may make a selection of a patient condition from the menu by activation of an input device such as a button or touch screen. In response to the selection by the operator, the device selects at least one configuration and configures the device with the configuration parameter.

In screen display 60, menu 62 includes four patient conditions: “cardiac,” “trauma,” “pediatric” and “medical.” These four patient conditions may be the most common patient conditions that the operator is likely to encounter. The “cardiac” condition may pertain to heart-related conditions, the “trauma” condition may pertain to conditions that result from trauma, and so forth.

In some embodiments of the invention, selection of one of the patient conditions may cause additional menus of patient conditions to be displayed. For example, selection of the “trauma” condition may cause the display to present a sub-menu of trauma-related patient conditions such as “bleeding,” “burns,” “fractures,” or other specific kinds of trauma. As a result, the configuration may be made specific to the condition experienced by patient 10.

The patient conditions listed in screen display 60 are exemplary, and the invention is not limited to these patient conditions. Other patient conditions presented in menu 62 may include “stroke,” “respiratory,” “abdominal pain,” and the like. Menu 62 includes an “other” option, that, when selected, may cause the device to display a menu including other patient conditions such as the patient conditions listed above.

Menu 62 may also include a “generic” option. Selection of the “generic” option may cause the device to select one or more generic configuration parameters and to configure the device with the generic configuration parameters. The device may also self-configure with the generic configuration parameters automatically. When an operator fails to make a selection from menu 62 after a predetermined time, for example, the device may automatically self-configure with the generic configuration so that the device may be ready to address general, non-specific emergencies.

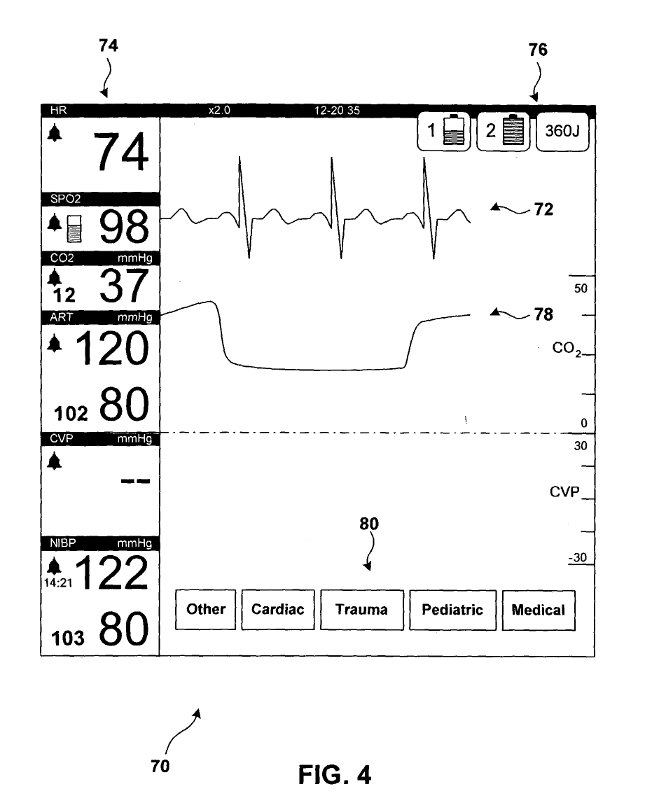

FIG. 4 is an example of a screen display 70 that illustrates an aspect of configuration. In particular, screen display 70 illustrates display of information that may be appropriate when an operator selects the “cardiac” patient condition on defibrillator 12. Screen display 70 includes, for example, an ECG waveform 72 sensed via electrodes 14 and 16, and information about the vital signs 74 of patient 10. Screen display 70 may further include information about the status 76 of the defibrillator 12, such as battery capacity, the current energy setting and the readiness of defibrillator 12 to deliver a defibrillation shock. In addition, screen display 70 may include information obtained from other medical devices, such as the blood pressure of patient 10 or carbon dioxide concentrations 78 sensed by a capnogram.

In addition to configuration of screen display 70, selection of the “cardiac” option may cause defibrillator 12 to self-configure in other ways as well. For example, defibrillator 12 may set alarm parameters or select a cardiac health care protocol, and may prompt the operator to perform a sequence of actions or report the status of treatment pursuant to the selected health care protocol.

Screen display 70 may also display a menu 80 of patient conditions. There are many circumstances in which an operator may wish to change the configuration of the medical device. For example, the operator may have selected the “cardiac” patient condition in error, or the operator may wish to change the configuration from a generic configuration to a configuration more suitable to the actual condition of patient 10. It is also possible that the operator may deem that a different configuration may be in order, such as when an operator originally determines that unconscious patient 10 may be suffering from a cardiac condition, but testing suggests that patient 10 may actually have suffered a stroke. In cases such as these, the operator may select a patient condition from menu 80. In response, the device selects one or more configuration parameters and self-configures with the configuration parameters.

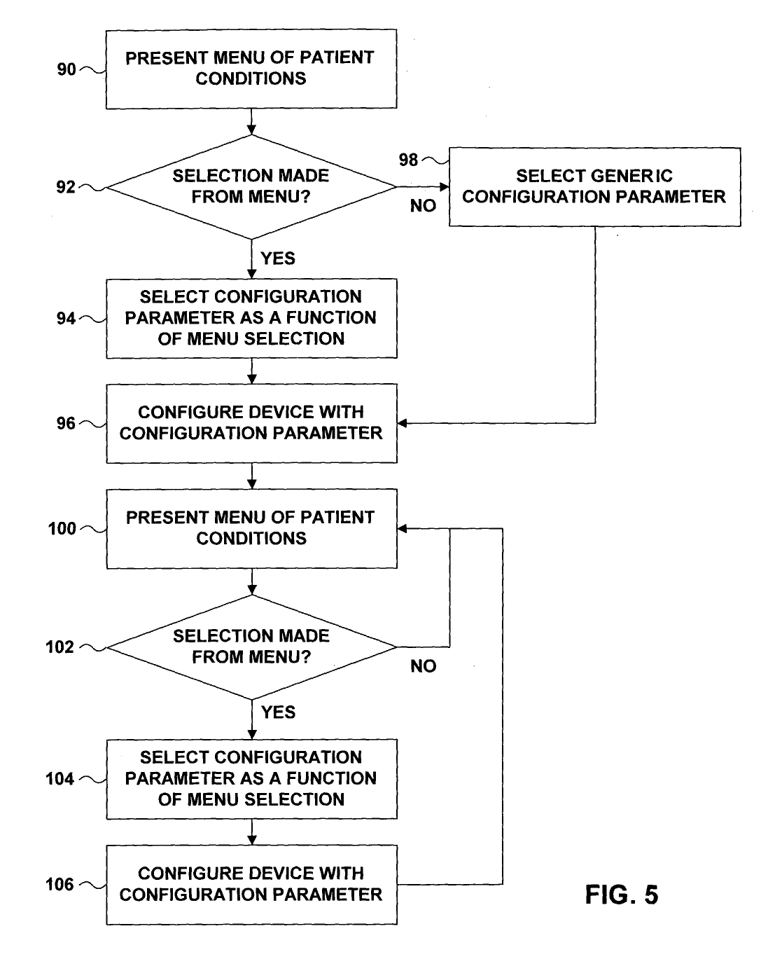

FIG. 5 is a flow diagram illustrating menu-driven medical device configuration techniques. The device presents a menu of patient conditions to an operator (90) and waits for an interaction with the menu by the operator (92). When the operator makes a selection of a patient condition from the menu, the device receives the selection and selects at least one configuration parameter as a function of the selected patient condition (94). The device self-configures with the configuration parameter (96).

In some circumstances, such as following the initial activation of the device and presentation of the menu (90), the device may wait for an interaction with the menu by the operator (92), but the operator may fail to make a selection. After a preselected waiting period, the device may select at least one generic configuration parameter (98) and may self-configure with the generic configuration parameter (96).

After the device has been configured, the device may allow the operator to change the configuration. In particular, the device may present a menu of patient conditions to an operator (100) and wait for an interaction with the menu by the operator (102). In the event the operator makes no selection, the device continues in the present configuration. When the operator makes a selection of a patient condition from the menu, the device receives the selection and selects at least one configuration parameter as a function of the selected patient condition (104). The device self-configures with the configuration parameter (106). Following self-configuration, the device may optionally present the same or a different menu of patient conditions to an operator (100).

The device may also self-configure as a function of the level of operator training. In some circumstances, the device may presents a menu of patient conditions to an operator and may also present a menu of operator training levels. The device may select one or more configuration parameters and may self-configure as a function of the patient condition and the selected operator training level.

Not all operators have equal training an experience. In comparison to a paramedic, a police officer may have had comparatively little training and experience in medical rescue techniques, and may need more assistance from the device when attending to the patient than would a paramedic. Accordingly, the device configuration may reflect the training and experience of the operator and provide the needed assistance. For example, when the operator selects an operator training level indicative of a low level of training, the medical device may present detailed prompts to perform a sequence of actions. The device may display less detailed prompts to an extensively trained EMT. Similarly, the device may display different information about patient status depending on the level of training of the operator.

The invention may offer one or more advantages. For example, menu-driven configuration may allow a medical device to be quickly and easily configured for a variety of medical conditions. An operator may configure the device to a general range of patient conditions, or to specific patient conditions. Moreover, an operator may configure the device while responding to the emergency, and may reconfigure the device in response to developments in the emergency. In addition, the device may include a default self-configuration with one or more generic configuration parameters, making the device ready to handle typical emergencies.

The preceding specific embodiments are illustrative of the practice of the invention. Various modifications may be made without departing from the scope of the claims. For example, the invention need not be embodied in a medical device such as defibrillator or medical monitor. The invention may be embodied in other devices that provide treatment, such as drug delivery devices. The invention may also be embodied in a stand-alone device that provides no diagnosis, monitoring or therapy, or a stand-alone device that receives information from other medical devices. The device may be small and easily portable, but the invention is not limited to application with small, portable devices.

Although the invention has been described in the context of emergency personnel configuring a medical device at the scene of an emergency, the invention is not limited to that context. The invention may be applied in other contexts as well, such as a hospital setting. A hospital emergency room, for example, may benefit from having a medical device that can be quickly customized to the needs of several patients through simple menu selections.

The invention may be embodied as a computer-readable medium that includes instructions for causing a programmable processor to carry out the methods described above. A “computer-readable medium” includes but is not limited to read-only memory, Flash memory and a magnetic or optical storage medium. The instructions may be implemented as one or more software modules, which may be executed by themselves or in combination with other software.

The instructions and the media are not necessarily associated with any particular computer or other apparatus, but may be carried out by various general-purpose or specialized machines. The instructions may be distributed among two or more media and may be executed by two or more machines. The machines may be coupled to one another directly, or may be coupled through a network, such as a local access network (LAN), or a global network such as the Internet.

The invention may also be embodied as one or more devices that include logic circuitry to carry out the functions or methods as described herein. The logic circuitry may include a processor that may be programmable for a general purpose or may be dedicated, such as microcontroller, a microprocessor, a Digital Signal Processor (DSP), Application Specific Integrated Circuit (ASIC), and the like. These and other embodiments are within the scope of the following claims.

1. A method comprising:
presenting a menu of patient conditions;
receiving a selection of a patient condition from the menu when an operator selects the patient condition from the menu;
selecting a configuration parameter; and
configuring a medical device with the configuration parameter.

2. The method of claim 1, wherein selecting a configuration parameter comprises selecting a configuration parameter as a function of the received selected patient condition.

3. The method of claim 1, wherein selecting a configuration parameter comprises selecting a generic configuration parameter when the operator fails to select any patient condition from the menu within a waiting period.

4. The method of claim 1, wherein the medical device is one of a defibrillator and a patient monitor.

5. The method of claim 1, wherein configuring the medical device with the configuration parameter comprises at least one of displaying patient information, setting a defibrillation energy level, selecting a protocol, and setting an alarm parameter.

6. The method of claim 1, further comprising:
presenting a menu of operator training levels to an operator;
receiving a selection of an operator training level from the menu; and
selecting at least one configuration parameter as a function of the selection of the operator training level.

7. The method of claim 1, further comprising:
presenting a second menu of patient conditions to the operator;
receiving a second selection of a patient condition from the second menu;
selecting at least one second configuration parameter as a function of the selection of the second patient condition from the second menu; and
configuring the medical device with the second configuration parameter.

8. A method comprising:
presenting a menu of patient conditions;
receiving from an operator a selection of a patient condition from the menu;
selecting at least one configuration parameter as a function of the selected patient condition; and
configuring a medical device with the configuration parameter.

9. The method of claim 8, wherein the menu of patient conditions is a first menu of patient conditions and the selection is a first selection, the method further comprising:
presenting a second menu of patient conditions as a function of the first selection;
receiving a second selection of a patient condition from the second menu; and
selecting the configuration parameter as a function of the second selection of the patient condition from the second menu.

10. The method of claim 8, wherein the menu of patient conditions is a first menu of patient conditions, the selection is a first selection and the configuration parameter is a first configuration parameter, the method further comprising:
presenting a second menu of patient conditions;
receiving a second selection of a patient condition from the second menu;
selecting at least one second configuration parameter as a function of the second selection of the patient condition from the second menu; and
configuring a medical device with the second configuration parameter.

11. A computer-readable medium comprising instructions for causing a programmable processor to:
present a menu of patient conditions to an operator;
receive a selection of a patient condition from the menu when an operator selects the patient condition from the menu;
select a configuration parameter; and
configure a medical device with the configuration parameter.

12. The medium of claim 11, wherein the instructions causing the processor to select the configuration parameter cause the processor to select a configuration parameter as a function of the received selected patient condition.

13. The medium of claim 11, wherein the instructions causing the processor to select a configuration parameter cause the processor to select a generic configuration parameter when the operator fails to select any patient condition from the menu within a waiting period.

14. The medium of claim 11, wherein the instructions causing the processor to configure the medical device comprise instructions causing the processor to perform at least one of displaying patient information, setting a defibrillation energy level, selecting a protocol, and setting an alarm parameter.

15. The medium of claim 11, the instructions further causing the processor to:
present a menu of operator training levels to an operator;
receive a selection of an operator training level from the menu; and
select at least one configuration parameter as a function of the selection of the operator training level.

16. The medium of claim 11, the instructions further causing the processor to:
present a second menu of patient conditions to the operator;
receive a second selection of a patient condition from the second menu;
select at least one second configuration parameter as a function of the selection of the second patient condition from the second menu; and
configure the medical device with the second configuration parameter.

17. A computer-readable medium comprising instructions for causing a programmable processor to:
present a menu of patient conditions;
receive from an operator a selection of a patient condition from the menu;
select at least one configuration parameter as a function of the selected patient condition; and
configure a medical device with the configuration parameter.

18. The medium of claim 17, wherein the menu of patient conditions is a first menu of patient conditions and the selection is a first selection, the instructions further causing the processor to:
present a second menu of patient conditions as a function of the first selection;
receive a second selection of a patient condition from the second menu; and
select the configuration parameter as a function of the second selection of the patient condition from the second menu.

19. The medium of claim 17, wherein the menu of patient conditions is a first menu of patient conditions, the selection is a first selection and the configuration parameter is a first configuration parameter, the instructions further causing the processor to:
present a second menu of patient conditions;
receive a second selection of a patient condition from the second menu;
select at least one second configuration parameter as a function of the second selection of the patient condition from the second menu; and
configure a medical device with the second configuration parameter.

20. A medical device comprising:
an output device to present a menu of patient conditions to an operator;
an input device to receive a selection of a patient condition from the menu; and
a processor to select at least one configuration parameter and to configure the medical device with the configuration parameter.

21. The device of claim 20, wherein the processor configures the medical device with the configuration parameter to perform at least one of displaying patient information, setting a defibrillation energy level, selecting a protocol, and setting an alarm parameter.

22. The device of claim 20, wherein the medical device is one of a defibrillator and a patient monitor.

23. The device of claim 20, wherein the medical device is a defibrillator, the device further comprising a charging circuit that stores energy in an energy storage device as a function of the configuration parameter.
